(19) United States
(12) Patent Application Publication
(10) Pub. No.: US 2026/0115153 A1
(43) Pub. Date: Apr. 30, 2026

(54) ANTI-ANXIETY COMPOSITION

(71) Applicant: Brent Ravnaas, Turtle Lake, ND (US)

(72) Inventor: Brian Ravnaas, Turtle Lake, ND (US)

(21) Appl. No.: 18/927,248

(22) Filed: Oct. 25, 2024

(51) Int. Cl. A61K 31/155; A61K 31/185; A61K 31/197; A61K 33/06

(52) U.S. Cl. CPC A61K 31/155; A61K 31/185; A61K 31/197; A61K 33/06

(57) ABSTRACT

An anti-anxiety composition includes 2 grams of Beta Alanine; 0.75 grams of agmatine sulfate; 0.75 grams of taurine; 0.75 grams of magnesium citrate; and 100 mg of l-theanine. A method for decreasing anxiety in a subject includes administering to the subject a drinkable liquid that includes 0.25-2 grams of Beta Alanine.

### CROSS REFERENCE TO RELATED APPLICATIONS

None

### BACKGROUND

The present disclosure relates to natural supplements, and more specifically, to an anti-anxiety composition.

According to the American Psychological Association, anxiety is a constant, uncontrolled feeling of worry. The Centers for Disease Control and Prevention (CDC) estimates approximately 12% of the U.S. adult population has regular feelings of worry, nervousness, or anxiety. When these feelings become strong enough and frequent enough to severely interfere with day-to-day activities, it’s possible you may be living with an anxiety disorder. Anxiety disorders are the most common form of mental illness in the U.S., with 40 million adults (19.1% of the population) affected annually.

With the global prevalence of anxiety on the rise, researchers are discovering that nutrition may play a more important role in mental health than previously thought. An interesting aspect of these discussions is the role natural supplements may have in easing anxiety.

### SUMMARY

An anti-anxiety composition according to one disclosed non-limiting embodiment of the present disclosure includes 0.25-2 grams of an amino acid.

A further embodiment of any of the foregoing embodiments of the present disclosure includes that the amino acid is powdered Beta Alanine.

A further embodiment of any of the foregoing embodiments of the present disclosure includes 0.25-2 grams of powdered agmatine sulfate.

A further embodiment of any of the foregoing embodiments of the present disclosure includes 0.25-2 grams of powdered taurine.

A further embodiment of any of the foregoing embodiments of the present disclosure includes 0.25-2 grams of powdered magnesium citrate.

A further embodiment of any of the foregoing embodiments of the present disclosure includes 0.25-2 grams of powdered taurine.

A further embodiment of any of the foregoing embodiments of the present disclosure includes 0.25-2 grams of powdered Beta Alanine; 0.25-2 grams of powdered agmatine sulfate; 0.25-2 grams of powdered taurine; and 0.25-2 grams of powdered magnesium citrate.

A further embodiment of any of the foregoing embodiments of the present disclosure includes 0.1-300 mg of powdered l-theanine.

An anti-anxiety composition according to one disclosed non-limiting embodiment of the present disclosure includes 2 grams of Beta Alanine; 0.75 grams of agmatine sulfate; 0.75 grams of taurine; 0.75 grams of magnesium citrate; and 100 mg of l-theanine.

A method for decreasing anxiety in a subject according to one disclosed non-limiting embodiment of the present disclosure includes administering to the subject a drinkable liquid that includes 0.25-2 grams of Beta Alanine.

A further embodiment of any of the foregoing embodiments of the present disclosure includes that the drinkable liquid further includes 0.75 grams of powdered agmatine sulfate.

A further embodiment of any of the foregoing embodiments of the present disclosure includes that the drinkable liquid further includes 0.75 grams of powdered taurine.

A further embodiment of any of the foregoing embodiments of the present disclosure includes that the drinkable liquid further includes 0.75 grams of powdered magnesium citrate.

A further embodiment of any of the foregoing embodiments of the present disclosure includes that the drinkable liquid further includes 100 mg of powdered l-theanine.

A further embodiment of any of the foregoing embodiments of the present disclosure includes that the drinkable liquid further includes 0.75 grams of agmatine sulfate; 0.75 grams of taurine; 0.75 grams of magnesium citrate; and 100 mg of l-theanine.

A further embodiment of any of the foregoing embodiments of the present disclosure includes that the drinkable liquid further includes 0.75 grams of powdered agmatine sulfate; 0.75 grams of powdered taurine; 0.75 grams of powdered magnesium citrate; and 100 mg of powdered l-theanine.

The foregoing features and elements may be combined in various combinations without exclusivity, unless expressly indicated otherwise. These features and elements as well as the operation thereof will become more apparent in light of the following description and the accompanying drawings. It should be appreciated that however the following description and drawings are intended to be exemplary in nature and non-limiting.

### DETAILED DESCRIPTION

An anti-anxiety composition may be prepared by pouring a packet of the anti-anxiety composition into water then mixing to form a drinkable liquid. Although a drinkable liquid is primarily disclosed, other drinkable, swallowable or edible forms may also be provided such as pills, gummies, etc.

In one embodiment, the anti-anxiety composition may include one or more of the following components:

Beta Alanine (an amino acid);

Taurine (an amino acid);

Magnesium (magnesium citrate);

L-theanine (an amino acid); and

Agmatine (agmatine sulfate).

In another embodiment, the anti-anxiety composition may include the combination of beta alanine and agmatine.

In another embodiment, the anti-anxiety composition may include the combination of beta alanine and taurine.

In another embodiment, the anti-anxiety composition may include the combination of beta alanine, taurine, and agmatine.

In another embodiment, the anti-anxiety composition may include the combination of beta alanine, taurine, agmatine and magnesium.

The anti-anxiety composition primarily utilizes amino acids, often referred to as the building blocks of proteins, which are compounds that play many critical roles in the body. Amino acids facilitate vital processes such as building proteins, hormones, and neurotransmitters and have been determined by applicant to have positive effects upon the treatment of anxiety.

Beta alanine (β-Alanine) is a naturally occurring beta amino acid, which is an amino acid in which the amino group is attached to the β-carbon instead of the more usual α-carbon for alanine. The IUPAC name for β-alanine is 3-aminopropanoic acid. Beta Alanine exerts a strong inhibitory effect on the central nervous system. Beta alanine may result in a type of skin tingling (Paresthesia) for a period of time after ingestion. Paresthesia is harmless, although some users may find it objectionable. How Beta Alanine exerts this effect is not entirely clear. Some research (The GABA uptake inhibitor beta-alanine reduces pilocarpine-induced tremor and increases extracellular GABA in substantia nigra pars reticulata as measured by microdialysis - PubMed (nih.gov)) states that Beta Alanine operates as a GABA reuptake inhibitor. A reuptake inhibitor may increase extracellular concentration of the neurotransmitter whose reuptake it blocks. Various pharmaceutical antidepressants are serotonin reuptake inhibitors. Another theory on beta alanine’s effect on mood is that it increases the expression of neutrophils, which lead to improved mood. However, this may be unlikely to occur in the approximately 15 minutes the anti-anxiety composition impacts the mood of the user. Beta alanine impairs the body’s taurine transporter, and a constant large concentration of beta alanine in serum can induce taurine deficiency, which comes with a variety of negative health consequences.

Taurine has a calming effect, is extremely important for health, and beneficially interacts with beta alanine. The impact of taurine- and beta-alanine-supplemented diets on behavioral and neurochemical parameters have been shown to provide antidepressant versus anxiolytic-like effects - PubMed (nih.gov). Beta Alanine may impair the body's taurine transporter, and a constant large concentration of Beta Alanine can induce taurine deficiency, which is offset by the inclusion of taurine.

Magnesium is an essential mineral that is crucial for the brain to regulate glutamate activity at a set of receptors called NMDA receptors, and as such can exert an anti-anxiety effect in people lacking adequate Mg levels. A substantial percent of the population does not receive enough magnesium from their diet (Study: Half of All Americans are Magnesium Deficient (pharmacytimes.com)) and the anti-anxiety composition effectively provides part of the recommended daily intake. The magnesium in the anti-anxiety composition is quickly available to the user and contributes to the success thereof.

L-theanine is an amino acid found naturally in tea.

Agmatine such as agmatine sulfate is a metabolite of the amino acid arginine produced in human bodies and present in the brain. Agmatines operate as an antagonist of glutamates activity at NMDA receptors. The shaking, sweating, anxiety (in extreme cases seizures) that can be associated with hangovers or alcohol withdrawal is the result of dysregulation and hyperactivity at NMDA receptors. Agmatine thus has a distinct calming effect to someone experiencing alcohol withdrawal (a severe hangover can manifest as subclinical withdrawal symptoms).

In another embodiment, the anti-anxiety composition may include:

0.25-2 grams of powdered Beta Alanine;

0.25-2 grams of powdered agmatine sulfate;

0.25-2 grams of powdered taurine; and

0.25-2 grams of powdered magnesium citrate.

In another embodiment, the anti-anxiety composition may also include: 0.1-300 mg of powdered L-theanine, and more specifically, 100 mg of powdered L-theanine.

In another embodiment, the anti-anxiety composition may include:

2 grams of powdered Beta Alanine;

0.75 grams of powdered agmatine sulfate;

0.75 grams of powdered taurine;

0.75 grams of powdered magnesium citrate; and

100 mg of powdered l-theanine.

The components may be each soluble in water and make an extremely effective anti-anxiety, anti-stress, mood boosting, calming product. Although water-soluble increments are disclosed, it should be appreciated that additional or alternative base solvents may be utilized as well as different forms such a solid pill form will benefit herefrom.

Although the different non-limiting embodiments have specific illustrated components, the embodiments of this invention are not limited to those particular combinations. It is possible to use some of the components or features from any of the non-limiting embodiments in combination with features or components from any of the other non-limiting embodiments.

The foregoing description is exemplary rather than defined by the limitations within. Various non-limiting embodiments are disclosed herein, however, one of ordinary skill in the art would recognize that various modifications and variations in light of the above teachings will fall within the scope of the appended claims. It is therefore to be appreciated that within the scope of the appended claims, the disclosure may be practiced other than as specifically described. For that reason, the appended claims should be studied to determine true scope and content.

What is claimed is:

1. An anti-anxiety composition, comprising:
0.25-2 grams of an amino acid.

2. The anti-anxiety composition as recited in claim 1, wherein the amino acid is powdered Beta Alanine.

3. The anti-anxiety composition as recited in claim 2, further comprising:
0.25-2 grams of powdered agmatine sulfate.

4. The anti-anxiety composition as recited in claim 3, further comprising:
0.25-2 grams of powdered taurine.

5. The anti-anxiety composition as recited in claim 4, further comprising: 0.0-25. grams of powdered magnesium citrate.

6. The anti-anxiety composition as recited in claim 2, further comprising:
0.25-2 grams of powdered taurine.

7. The anti-anxiety composition as recited in claim 2, further comprising:
0.25-2 grams of powdered Beta Alanine;
0.25-2 grams of powdered agmatine sulfate;
0.25-2. grams of powdered taurine; and
0.25-2 grams of powdered magnesium citrate.

8. The anti-anxiety composition as recited in claim 7, further comprising:
0.1-300 mg of powdered l-theanine.

9. An anti-anxiety composition, comprising:
22 grams of Beta Alanine;
0.75 grams of agmatine sulfate;
0.75 grams of taurine;
0.75 grams of magnesium citrate; and
100 mg of l-theanine.

10. A method for decreasing anxiety in a subject, the method comprising:
administering to the subject a drinkable liquid that comprises 0.25-2 grams of Beta Alanine.

11. The method as recited in claim 10, wherein the drinkable liquid further comprises:
0.75 grams of powdered agmatine sulfate.

12. The method as recited in claim 10, wherein the drinkable liquid further comprises:
0.75 grams of powdered taurine.

13. The method as recited in claim 10, wherein the drinkable liquid further comprises:
0.75 grams of powdered magnesium citrate.

14. The method as recited in claim 10, wherein the drinkable liquid further comprises:
100 mg of powdered l-theanine.

15. The method as recited in claim 10, wherein the drinkable liquid further comprises:
0.75 grams of agmatine sulfate;
0.75 grams of taurine;
0.75 grams of magnesium citrate; and
100 mg of l-theanine.

16. The method as recited in claim 10, wherein the drinkable liquid further comprises:
0.75 grams of powdered agmatine sulfate;
0.75 grams of powdered taurine;
0.75 grams of powdered magnesium citrate; and
100 mg of powdered l-theanine.
